(19) United States
(12) Patent Application Publication
(10) Pub. No.: US 2022/0409404 A1
(43) Pub. Date: Dec. 29, 2022

(54) System and Method for an Improved Redundant Crossfire Circuit in a Fully Integrated Neurostimulation Device and Its Use in Neurotherapy

(71) Applicant: Regents of the University of Minnesota, Minneapolis, MN (US)

(72) Inventors: Zhi Yang, Minneapolis, MN (US); Anh Tuan Nguyen, Minneapolis, MN (US); Diu Khue Luu, Minneapolis, MN (US)

(21) Appl. No.: 17/849,534

(22) Filed: Jun. 24, 2022

Related U.S. Application Data
(60) Provisional application No. 63/214,623, filed on Jun. 24, 2021.

(51) Int. Cl. A61F 2/72; G05F 3/26; H03M 1/66; A61F 2/58

(52) U.S. Cl. CPC A61F 2/72; G05F 3/262; H03M 1/66; A61F 2/583; A61F 2002/5061

(57) ABSTRACT

A neurostimulator incorporating a novel chip design that uses the principle of redundant signal crossfiring to overcome electronic component mismatch error in general and transistor mismatch error in particular, to yield superior quality neurostimulation signal generation, useful in enhancing the bidirectional human-machine interface in prosthesis operation for the restoration of somatosensation for an amputee.

### CROSS-REFERENCE TO RELATED APPLICATIONS

This application is based on, claims priority to, and incorporates by reference in its entirety for all purposes, U.S. Provisional Application Ser. No. 63/214,623, filed Jun. 24, 2021, and entitled, “SYSTEM AND METHOD FOR AN IMPROVED REDUNDANT CROSSFIRE CIRCUIT IN A FULLY INTEGRATED NEUROSTIMULATION DEVICE AND ITS USE IN NEUROTHERAPY.”

### STATEMENT ON FEDERALLY SPONSORED RESEARCH

This invention was made with government support under Agency Grant Number 1845709 awarded by the National Science Foundation, and Agency Grant Number 1R21NS111214-01 awarded by the National Institute of Health. The government has certain rights in the invention.

### FIELD OF THE INVENTION

This invention generally concerns systems and methods for neuromodulation, and, more specifically, concerns miniaturized neurostimulators in integrated circuits that convert and exploit the problem of mismatch error into an effective means of achieving super-resolution signal accuracy in neuromodulation.

### BACKGROUND OF THE INVENTION

Electrical stimulation has been used in research and in therapeutics for probing neural circuitry and identifying networks of neurons for many years. Despite its extensive use, the mechanism of electrical stimulation on the nervous system remains poorly understood, and the results of applying novel approaches to such therapeutic stimulation cannot be predicted with a reasonable degree of certainty by those of ordinary skill in the art due to the uncertainty of the outcome prior to pre-clinical experimental research on animal models and clinical experimental development on human subjects. There exists a need for an integrated hardware and software platform system that can sense, analyze, and modulate neural signals, and to define, incorporate, and transmit neural feedback-influenced electrical signals in order to improve electrical stimulation outcomes. Currently available or known electrical stimulation devices do not adequately sense nerve neural signals, being unable to satisfactorily overcome the fact that nerve neural signals are smaller than signals measured in the brain, meaning that existing recording technology is inadequate for resolving nerve neural signals from signals measured in the brain, which constitute sources of signal noise.

Signal Noise. Noise, in the context here of signal processing, is defined as a general term for unwanted (and, in general, unknown, unknowable, or unpredictable) modifications that a signal may suffer during capture, storage, transmission, processing, and conversion of the signal for purposes of recording and analyzing the signal of interest. This problem is made even worse when a prior art neurostimulation device is a non-dermal penetrating type of cuff electrode. When penetrating or intraneural electrodes are used to address this problem, nerve signal quality is improved at early time points, but the signal decays over time due to foreign body host response processes (e.g. inflammatory responses) and due to the micro-motion of the position of the tethered electrodes, in relation to the surrounding soft tissues into which the electrodes are embedded. Additionally, there is the inherent problem that whenever electrical stimulation is applied, there are large, sustained noise artifacts. Prior art attempts to address this have been through efforts at blocking or blanking out the noise, rather than subtracting out the noisy signals, thereby losing neural responses that may be measured locally with greater accuracy. An integrated approach as utilized in the present invention for developing a superior neuromodulation device therefore will comprise the four strategies of: (1) developing ultra-low noise hardware technology for sensing neural signals, most preferably (but not exclusively) without requiring electrode penetration into a nerve; (2) developing a deep neural network-based algorithm to analyze and decode signals; (3) developing hardware technology for both selective amplification of signals of interest and for removing stimulation signal residual charges and noise artifacts to support simultaneous recording and stimulation of a nerve; and (4) developing prediction models of therapeutic outcomes, based on stimulation parameters and neural signals. The present invention will, in its most preferred embodiment, play a major part in signal analysis and decoding, operating within the overall architecture of this four-part integrated approach.

Poor signal-to-noise ratio (SNR) of neural signals in nerves. Epineural electrodes, such as cuffs, provide a robust and stable interface for recording whole-nerve neural activity, but the electrical isolation of the epineurium/perineurium reduces the magnitude of signals, resulting in a low SNR. In particular, the biological noise in epineural electrodes can significantly distort recordings and even bury the neural signals, if the precision of the recorder is not sufficiently high. Post-signal processing to remove biological noise often creates new noise artifacts that can be confounded with nerve signals. Due to different chemical compounds, cell tissue environments, and tissue responses to implants, the electrode-tissue interface noise can be very different from a measurement in a bench testing apparatus. Several models and more recent measurement data suggest that a large portion of electrode noise is non-Ohmic (Johnson-Nyquist noise, or thermal noise), which is the electronic noise generated by the thermal agitation of the charge carriers (usually the electrons) inside an electrical conductor at equilibrium, which happens regardless of any applied voltage. For such non-Ohmic noise, the sensing electronics can play a role in reducing the electrode noise. The amount of electronics noise in nerve recordings can be quite large due to elevated filter noise. For example, when recording a nerve in the abdomen, the constant motion of viscera causes large amplitude-motion artifacts. To avoid saturating electronics, an add-on analog filter with a corner frequency at least ten times higher than the artifact frequency is required. This ten-fold requirement increases the analog filter noise in the neural signal band up to tens of μV and beyond. Therefore, it can be seen that the battle to improve neural recording signal quality goes beyond the optimization of individual noise components.

Inabilities to extract signals of individual fascicles and inabilities to develop chronic stability. Noninvasive nerve interface approaches provide small signals from a highly limited number of electrodes. Current technologies have demonstrated that recordings of compound nerve action potentials (CNAPs), mostly from exposed nerves, are sufficiently greater than the background noise activities, but CNAPs constitute such a volley of synchronous action potentials that it is not possible to deconvolute them into individual fiber activities, which is required to perform neural decoding. Spontaneous or less-synchronized neural activities from a nerve are much smaller than CNAPs, and where it is particularly true of cuff electrodes, it is seen that such signals are further attenuated by the physical presence of the nerve sheath. For example, it has been confirmed in the prior art that after using neuromuscular blockers, surface canine vagus nerve signals are in the range of microvolts to tens of microvolts for de-sheathed nerves, but are even smaller (estimated at less than 10 μV) when the epineurium was intact during surface recordings. A nerve is an enclosed, cable-like bundle of many myelinated nerve fibers, structured as sheaths: the endoneurium is a layer of connective tissue around the myelin sheath of each fiber. The endoneuria with their enclosed nerve fibers are bundled into nerve fascicles, and each fascicle is within its own protective sheath called a perineurium. Multiple fascicles are bundled within yet another sheath, the epineurium. These multiple protective sheaths isolate fibers, making it extremely hard to record and then modulate signals on individual fibers from the outside of the nerve. Invasive intraneural electrodes may produce detectable signals at early time points, but signal decay over time due to tissue response has remained a problem. Initial insertion of the electrodes causes an early host immune system foreign body response leading to tissue encapsulation, with extensive tissue damage resulting from the micro-motion of the tethered electrodes relative to the soft tissue. Due to a lack of chronic stability, intraneural electrodes pose a significant long-term usage challenge. Therefore, there is still a need to pursue non-penetrating neural interfaces with significantly reduced noise so as to be able to accurately record small nerve signals, which is a necessary prerequisite to therapeutic neuromodulation.

Inability to record microvolt-level nerve activity in the presence of stimulation pulses applied to the same nerve. During stimulation, electrode-generated stimulation artifacts often produce a shift in the differential mode signal and the common mode signal, both of which need to be properly rejected. Subtraction of the stimulation artifactrequires compensating for the frequency-dependent amplitude and phase of the transfer function. This subtraction process cannot be done as a matter of routine because the process requires a very high precision amplifier buffer to store the collection of superimposed artifacts and the collected neural signals. As noted, in brain recording experiments, the amplitude of neural signals (spikes and field potentials) is seen to be around a few hundred μV, but the amplitude of stimulation artifacts can reach as great as several Volts. To deal with a signal amplitude that can be less than 10 μV, there is a requirement for higher precision electronics capable of carrying out linear amplification. Every extra bit of increased precision of this type requires a 4-fold increase in the supplied power, which is consistent with the thermal slope in commercial analog electronics. As a result of this phenomenon, a commensurate upgrade of present-day brain technologies that could support simultaneous recording and stimulation on a peripheral nerve would require a multi thousand-fold increase in power. A power source capable of such power will be relatively large, bulky, and highly undesireable from a patient's point of view, possibly even leading to patient non-compliance with use of the device.

Mismatch error. A central problem in the development of neurostimulator designs for electrical stimulation of neurons is referred to by those of ordinary skill in the art as that of mismatch error. In a signal processing system several operations or computations are performed on a signal in different stages sequentially. Each of these operations seeks to emphasize a wanted component or property of the signal of interest, without adding too much, or too many, unwanted extra components. These unwanted extra components are now known to be due to the non-idealities of a given circuit's implementation compared to the circuit's specified operation. Circuit non-idealities can be divided into two groups: random errors; and systematic errors.

In this field, random errors are the result of the randomly-determined, or stochastic, nature of many physical processes. The stochastic behavior of charge carriers in a conductor, for instance, results in various types of noise signals, and the stochastic nature of the physical phenomena that take place during the manufacturer's fabrication of integrated circuits results in a random variation of the properties of the fabricated on-chip devices and furthermore results in mismatches between identically designed devices.

Systematic errors occur because a typical circuit implementation only approximates an ideal signal processing operation to a limited extent. These errors are caused, for instance, by the non-linear operating characteristics of devices or by the influence of parasitics (unwanted resistance, capacitance, or inductance in a circuit element) in the signal path or device structure.

The effect of these non-idealities may be of different kinds. The non-ideality of noise signals limit the minimal signal that can be processed with the system. The non-ideality of device mismatch limits the accuracy of the circuit behavior and again limits the minimal signal or energy that is required to execute meaningful signal operation functions. For linear systems, the non-linearities of devices generate distortion components of the signals or modulate unwanted ‘noise’ signals into the used signal band. This typically limits the maximal signal that can be processed correctly.

The circuit designer of ordinary skill in the art has heretofore attempted to reduce the effect of such distortion non-idealities by using small modulation indices for thebias signals. By using large device sizes, the impact of mismatch is lowered and by using low impedance levels, the thermal noise signals are reduced. Such measures have, however, very important consequences on the power consumption and operational speed of the system. Therefore, the quality of a circuit realization is evaluated from the obtained accuracy, noise level, or linearity relative to the used power and the speed of operation, all in an effort to achieve, for a given speed, an optimized balance of best performance with minimal power consumption.

Transistor mismatch. In the specialized circuit case of transistor mismatch, the circuit designer of ordinary skill can reduce the effect of distortion non-idealities by the use of small modulation indices for the bias signals; so by using large device sizes, the impact of mismatch is lowered, and by using low impedance levels, the thermal noise signals are reduced. These measures have, however, very important consequences on the power consumption and operation speed of the system. Therefore, the quality of a circuit realization is evaluated from the obtained accuracy, noise level or linearity relative to the used power and the speed of operation. The designer would try to achieve, for a given speed, the best performance with minimal power consumption.

Inherent transistor mismatch. Any two identically designed devices, here transistors, on an integrated circuit have random differences in their behavior and show a certain level of random mismatch in the parameters which model their behavior. This mismatch is due to the randomly determined, or stochastic, nature of physical processes that are used to fabricate such devices. Mismatch is thus the process that causes time-independent random variations in physical quantities of identically designed devices. For example, the mismatch of two complementary symmetry metal-oxide silicon transistors (CMOS), that are identical in their manufacture, is characterized by the random variation of the difference in their threshold voltage V_{T0}, their body factor y and their current factor β. For technologies with a minimal device size larger than typically 2 μM, a widely accepted and experimentally verified model for these random variations is a normal distribution with mean equal to zero and a variance dependent on the gate-width W and gate-length L and the mutual distance D between the devices. The characterization of transistor mismatch is a tedious process which requires a very large measurement effort. The design, realization, and validation of the measurement set-up, the acquisition of the experimental data and the statistical processing of the data have to be performed with great care to avoid errors and systematic effects. Moreover, when migrating towards sub-micron and deep-sub-micron technologies, the standard mismatch models have to be checked for their validity; if necessary, the effects of the short or narrow channel effects have to be accounted for in model extensions. A general relation can be derived for the minimal power consumption of a signal processing system due to the effect of the mismatch in the components. Transistor mismatch (as well as the phenomenon of thermal noise) are both random processes and therefore put a limit on the smallest signal that can be processed in a circuit; both phenomena will impose a minimum power consumption to achieve a certain drive, specification, accuracy, and speed. Prior art knowledge in the field of transistor mismatch held that such mismatch puts a fundamental limitation on the maximal total performance of analog signal processing systems, since the Speed-Accuracy/Power ratio is fixed by technological constants that constrain the ability to express the theoretical matching quality of the technology as a device.

Exemplary prior art efforts and attempts to control and reduce mismatch are discussed in “Implications of transistor mismatch on analog circuit design and system performance”, https://www.ee.columbia.edu/˜kinget/papers_files/chapter3.pdf, the entire disclosure of which is incorporated herein by reference, and which readily shows the great degree of challenge and importance placed on the problem of reducing mismatch. The origins of transistor mismatch, on the other hand, are linked to the given device structure, and device physics, and to the fabrication technology of integrated circuits. Device mismatch originates from the stochastic nature of physical processes used for the industrial fabrication of devices, such as ion implantations, diffusions, or etching. The device structure using a channel in a doped material and its operation by modulating the channel resistance result in random fluctuations of the device's properties and operation. For integrated circuit technologies that are fabricated and used today, these physical limitations are very fundamental and device mismatch is unavoidable. From this perspective, the limits imposed by device mismatch are restricted to signal processing systems realized using integrated circuits. As such, they are of course very important in the quest for minimal power consumption in integrated circuits. Prior art attempts to cope with the above described challenges include the following fields.

Deep neural networks to process nerve neural signals. Recording nerve neural signals with cuff electrodes is an important milestone towards developing a high-performance, minimally invasive neural interface. The thrust of this is to develop tools to analyze and understand the observed neural signal recordings. Differing from a brain single-unit recording which contains activities of a few neurons, a cuff electrode records neural signals from a nerve bundle of thousands of axons, where the recorded signals can vary in shape and in pattern, and are characterized in having poorer signal to noise ratios. Therefore, methods commonly used to process brain signals (for example, spike sorting, firing interval engineering, and rate based codings) will be less effective in processing nerve neural data. The ability to separate weak neural signals from background noise is crucial in nerve signal processing. Signal detection is preferably accomplished in a preferred embodiment of the invention by using a modified deep variational autoencoder (VAE) means for signal detection. An exemplary deep VAE consists of sequentially connected encoder and decoder networks, where the encoder learns a class label y and a probability distribution of the code z with stochastic variables of the input data x, and the decoder aims to reconstruct the input based on the class label and the code. Use of such a deep VAE means may enable the development of a large-scale, well-annotated nerve dataset, as well as a thorough exploration of inputted signals and noise, and the generation of representations of the collected and inputted signals and noise, all through the use of the deep VAE, which in turn enables the enforcement use of a de-noising criterion such that the noise will be maximally removed. After such a training phase, the deep VAE will be able to de-noise the received data in a subject or patient and hence to improve signal detection. The present invention's novel use of an improved and novel VAE is performed in combination with a novel dataset and a novel de-noising algorithm.

Datasets. Deep learning algorithms rely on large-scale, well-annotated datasets to achieve a superior performance. For example, ImageNet, a large-scale visual database designed for use in visual object recognition software research, contains over 14 million hand-annotated images, and is considered by those of ordinary skill in the art as having enabled a revolution in deep learning. The database of annotations of third-party image URLs is freely available directly from, for example, ImageNet at https://www.image-net.org. In the present invention, to bridge deep learning and neural signal processing, a dataset similar to the taxonomy and annotation strategy of ImageNet is first constructed, according to procedures and processes well known to those of ordinary skill in the artificial intelligence (AI) arts. The dataset then is used for developing neural signal processing algorithms.

Dataset generation. Cuff electrode data are a data summation from both filtered intraneural signals (signals within the nerve) and from noise arising out of external sources. Normally, cuff electrode data (both signals and labels) are not available for learning algorithms, especially for supervised learning, to record high quality, multi-site intraneural signals, and must be generated by the user or practitioner as part of the practice of the invention as claimed. The inventors have now built a cuff electrode dataset based on intraneural signals. First, a finite element model of epineurium signals is developed to simulate cuff electrode neural signals based on multi-site intraneural signals. To this database is then added noise that has been segmented from cuff electrode recordings, and the procedure is repeated with data from different electrodes and data from experimentally-derived and from third party-sourced animal model preparations. This yields a dataset derived from a sufficient set of experiments that the dataset can support the development of supervised learning algorithms to process neural signals.

Deep learning de-noising. Mathematically, the data representation process with the deep VAE can be expressed as:

p_{θ}(x, y, z)=p_{θ}(x|y, z)p(y)p(z)

where p_{θ}(x|y, z) quantifies how the observed values of x are related to the latent random variables y and z, and p(y), p(z) represent a known prior distribution of the latent variables y and z. Given this representation model, the posterior distribution p_{θ}(y|z, x) can be used to infer y, z and to find parameters θ that maximize the marginal likelihood p_{θ}(x). To approximate the intractable p_{θ}(y|z, x) a decoding distribution q_{Φ}(y|z, x) is modeled by learning the parameters Φ from the data. Next, one considers a 1-D time-series input xₜ={X_{t-T1}, . . . , X_{t+T2}}, where X represents a single-electrode recording and [t−T₁, t+T2] is a temporal scanning window. The binary classification label (i.e., either neural signals or noise) at time t is denoted as a one-hot vector yₜ, and the corresponding latent variables are represented as zₜ. The preferred embodiment of the deep VAE of the present invention models a joint distribution according to pe(xt I yt, zt) factorized as pθ(xₜ, yₜ, zₜ)=p_{θ}(xₜ|yₜ, zₜ)p(yₜ)p(zₜ). For the decoder model, the present invention uses p_{θ}(xₜ|zₜ, yₜ)=N(μ_{θ}(zₜ, yₜ), σ²_{θ}(zₜ, yₜ)I); and for the encoder model, relying on the theory of variational inference to approximate the intractable posterior, p_{θ}((z|x, yₜ) with a tractable auxiliary distribution a_{Φ}(zₜ|xₜ, yₜ)=N(η_{Φ}(xₜ, yₜ), σ²_{Φ}(xₜ)I). In the supervised case with an annotated dataset, the label yₜ is observed, allowing the parameters θ and Φ to be optimized by maximizing the extended variational lower bound as expressed as:

log p_{θ}(xₜ, yₜ) [symbol] _{qϕ}(zₜ|xₜ, yₜ)[log p_{θ}(xₜ|yₜ, zₜ)+log p_{θ}(yₜ)+log p_{θ}(zₜ)−log q_{Φ}(zₜ|xₜ, yₜ)]= [symbol] L_{L}(xₜ, yₜ).

For de-noising a nerve recording, the user injects the cuff electrode noise ε into xₜ to synthesize cuff recordings ^{˜}xᵢ=xᵢ+ε, where the noise is picked from the cuff data.

Simultaneous recording and stimulation on a peripheral nerve. Proof of concept of electroceuticals requires integrating the stimulation function and performing personalized adaptive neural modulation therapies based on neural feedback. Another challenge that the invention had to overcome is that nerve neural signals are relatively very weak, and thus they are vulnerable to the stimulation noise artifacts. To reduce such noise artifacts, a key feature of the novel invention as claimed is that of the novel redundant crossfire (RXF) stimulator design, based upon redundant sensing scientific theory.

Redundant sensing. Redundancy is a fundamental characteristic of many biological processes such as those in the genetic, visual, muscular, and nervous systems; yet its underlying causative driving mechanism is not well understood. A complete discussion of the phenomenon of redundancy is set forth at A Bio-inspired Redundant Sensing Architecture, accessible at http://papers.nips.cc/paper/6564-a-bio-inspires-redundant-sensing-architecture.pdf the entire disclosure of which is incorporated herein by reference. The present invention utilizes the redundancy inherent in materials engineering to enhance the accuracy and precision aspects of the system, by focusing on the application of the phenomenon of redundancy to reduce stimulation signal residual charge, and thus reduce the effects of stimulation noise artifacts. In the invention's use of redundant sensing, each entry of information can be represented by a plurality of distinct configurations or microstates, and there is a distinct subset of such microstates that will allow linear representation of the entries of information, and such a set or subset will not be bounded by the classic Shannon limit (see C. E. Shannon, The Mathematical Theory of Communication, by Claude E. Shannon and Warren Weaver. University of Illinois Press, 1964, the entire disclosure of which is incorporated herein by reference) when processed according to the practice of the present invention. The invention's identification of an optimized subset is an NP-incomplete problem, but it is possible to find a sub-optimal solution with sufficient efficiency to obtain a well-operating final complete embodiment of the invention. For example, in the case where a target stimulus signal is a 100 μA biphasic current, with a 6-bit resolution in amplitude, the anodic and cathodic branches will have up to a 3% mismatch depending on electrode conditions and clock jitter. Thus, the mismatch current is 100 μA×(3%+½⁶)=4.5 μA, which represents the cause of the resulting residual charge and stimulation noise artifacts. It can be seen from this example that a given amount of mismatch is stimulus dependent, time variant, and sensitive to electrode-electrolyte offset, thus posing a significant challenge to effective reduction of residual charge and stimulation noise artifacts, without having to resort to the prior art strategies of increasing the amount of power consumed or increasing the size of the neurostimulation device, both of which are product design strategies that ultimately produce a finished device of poor ergonomics and disappointing user satisfaction, possibly even resulting in patient noncompliance.

RXR stimulator. Based on the redundant sensing strategy, the present invention comprises an redundant crossfire, or RXF, stimulator, wherein the outputs of two or more independent stimulation channels with a current-digital-to-analog converter (IDAC) output driver will effectively form a redundant sensing structure. The sensed redundancy is exploited to fine tune and achieve precise matching between the anodic and cathodic stimulation currents, thus suppressing the residual charge and stimulation noise artifacts.

Accordingly, a need exists for a solution to at least one of the aforementioned challenges. The present invention addresses the challenges of the shortcomings of the prior art in attempting to solve the problems in circuit engineering described above, by designing a high-resolution constant-current stimulator (CCS) in integrated circuits without incurring penalties such as relatively large silicon area and/or high degree of power consumption because of the phenomenon of electronic component mismatch error in general, and transistor mismatch error in particular. Studies have shown that the phenomenon of random mismatch error is one of the major factors in limiting circuit design's effective resolution of such problems, especially when scaling down the circuits to submicron CMOS processes. Arising from random variations in the lithography process that are beyond the designers' control, mismatch error is the scourge of high-resolution analog IC designs. Nearly all neurostimulator designs are affected because mismatches appear in any type of component on an integrated circuit (IC) such as transistors, diodes, resistors, and capacitors. Current-controlled stimulators (CCS) are primarily affected by transistor mismatches, while voltage-controlled stimulators (VCS) and switched-capacitor stimulators (SCS) are primarily affected by transistor and capacitor mismatches. Mismatches influence the circuit's ability to produce accurate current/voltage output and synchronously match negative and positive stimulation phases, leading to charge imbalance problems. The work and resources involved in compensating for these errors requires extra calibration circuits, which in turn add complexity, area, and power.

Exploitation of the phenomenon of excessively large mismatches. All previous works in the literature have employed calibration circuits or chip layout techniques to attempt to remove or compensate for random mismatch error. Here, in the present invention as claimed, we disclose an entirely different approach to address this challenge, not by avoiding or compensating the mismatch error, but by embracing and exploiting excessively large mismatches to achieve super- resolution over 10-fold beyond the intrinsic resolution of the design. The redundant crossfire, or RXF, circuitry and method involves combining (i.e., crossfiring) the output of two or more current drivers to form a redundant structure. In the presence of large random mismatch errors (10-20%), such resultant redundant structure may be configured to generate the output that has an effective resolution that is many times above the Shannon limit as determined by physical constraints, including the number of unit-transistors. RXF in the present invention as claimed is a practical derivation of the theoretical framework that includes RS and RS-based super-resolution, wherein information redundancy is elegantly engineered and manipulated to boost system resolution. Furthermore, by handling extremely large mismatches, the present invention now makes it possible to implement high-resolution analog circuits in deep submicron technologies.

High-resolution, fully-integrated neurostimulators are at the core of many implantable or wearable devices. The neurostimulator's function is to generate stimulationpulses with accurately controlled parameters such as pulse-width, current amplitude, and frequency. It is a well-established method for interfacing with neural circuits well known to those of ordinary skill in the art. Proper uses of neurostimulation potentially offer a viable conduit to relay information to the human brain, and neurostimulation is widely used in neuroprosthesis applications to augment or replace missing or degraded human sensory functionality. Uses can include retinal implants for restoring vision, cochlear implants for restoring hearing, and prosthetic limbs for restoring somatosensation.

The present invention demonstrates a specific, most highly preferred application of a novel neurostimulator chip, in a prosthetic limb neuroprosthesis system, for the restoration of somatosensation for an amputee. A similar design can be adopted in a wide range of neuromodulation applications where the stimulator is the essential component of a bidirectional human-machine interface.

In a preferred embodiment of the present invention, a novel circuit, conceived of to be a component of one or more integrated circuits, is employed in order to utilize a redundant crossfire (RXF) technique in the design of neurostimulators, with super-resolution accuracy. The technique derives from the redundant sensing circuit architecture known to those of ordinary skill in the art. See Luu et al. (2019). Achieving Super-Resolution with Redundant Sensing. IEEE Transactions on Biomedical Engineering, 66(8), 2200-2209. (2019), the entire disclosure of which is incorporated herein by reference. There, the aim was to achieve engineering information redundancy, built into the system's architecture, in order to exploit the phenomenon of random transistor mismatch, and to thereby enhance the overall effective resolution capability of the device. The application of RXF in the present invention involves combining the RXF (i.e., crossfiring) outputs of two or more current drivers in order to form a redundant structure. See Nguyen et al. (2021). Super-Resolution in Neurostimulator Design by Exploiting Transistor Mismatch. IEEE Journal of Solid-State Circuits, 56(8), 2452-2465. (August 2021), the entire disclosure of which is incorporated herein by reference. When properly configured, this novel redundant structure can produce accurate current pulses with an effective super-resolution that is beyond the limitation commonly permitted by the physical constraints. Notably, and unlike any previous works, the novel circuitry of the device of the present invention unexpectedly and surprisingly achieves high accuracy by directly exploiting random transistor mismatch with an excessively large mismatch ratio of 10-20%, an outcome which cannot have been predicted with a reasonable degree of success.

In one most preferred embodiment of the present invention, effectiveness is significantly increased by, for example, incorporating a neurostimulator chip characterized in that each of a plurality of current drivers has a 5-bit current digital-to-analog converter (IDAC) and two 4-bit current multipliers. By crossfiring two drivers, it is feasible to achieve an effective super-resolution of 9.75 bits in a 1.1 mA full-range or 27× the ideal intrinsic resolution of each IDAC. The performance boost is verified through benchtop measurements of fabricated chips, and is consistent with the Monte Carlo (i.e. a broad class of computational algorithms that rely on repeated random sampling to obtain numerical results, where the underlying concept is to use randomness to solve problems that might be deterministic in principle) simulations that were run, demonstrating that the proposed technique is robust and dependable. A most highly preferred application of the fabricated chip here is to deliver neuro-feedback to a human amputee through peripheral nerves, where the amplitude of stimulation pulses is being accurately controlled in order to encode the intensity of tactile response.

### SUMMARY OF THE INVENTION

In summary, the invention, in its most preferred embodiment, is a neurostimulator system, comprising at least one digital-to-analog converter configured to receive an analog peripheral nervous system electrical signal from a subject or a patient, and to convert the analog signal into a corresponding digital signal; at least two current mirror circuits configured to receive digital electrical signals from the digital-to-analog converter and to provide mirrored current to at least two additional circuit components; at least two or more current drivers, at least one being an anodic output current driver, and at least one being a cathodic output current driver, the drivers being configured to scale the current signals received from the mirror circuits by a multiplying factor, and further configured to driving the constant current to at least one output electrode; wherein the outputs of the two or more current drivers are configured so as to create a combined, crossfiring, output of the current drivers that produces a redundant sensing structure, that in turn produces accurate current pulses that have the property of having an effective super-resolution accuracy beyond the ordinary limitations that are imposed by the predicted inherent physical constraints of materials in the system. The redundant sensing structure is configured so as to achieve a super-resolution signal accuracy outcome by applying the effects of random mismatch error function to the system, with the proviso that mismatch avoidance and mismatch compensation functions are not applied in achieving such super-resolution signal accuracy outcomes. In a most highly preferred embodiment, the random mismatch error function is configured so as to select and tune transistor size to achieve a desired mismatch ratio of 10% to 20%. Preferably, the system additionally comprises an on-chip timing generator, and additionally comprises both on-chip and off-chip components that are each configured so as to retrievably store calculated optimal transistor configurations obtained through foreground calibration, and which configurations can be retrieved and read by the on-chip timing generator so as to produce signal output with super resolution accuracy. Such an on-chip component is preferably a memory chip component of the system, whereas the off-chip component is a look-up table component of the system. The system additionally comprises an external controller configured so as to ensure charge-balancing that is achieved by digital compensation for residual mismatch between the anodic and cathodic currents. This charge balancing capability of the system is further characterized as being able to finely level charge balancing within the system, or to coarsely level charge balancing within the system. The system is configured so as to modulate the neurostimulation intensity of the cross firing redundant signal output to create various levels of somatosensorial signal outputs of from light to strong touch in real time in a neuroprosthesis device. The described random mismatch error function is configured so as to select and tune resistor size or to tune capacitor size or to tune diode size to achieve a desired mismatch ratio. A preferred embodiment of the system is configured so as to apply the effects of a random mismatch error function in the system, in turn configured so as to be applied to extremely large mismatch sets to achieve super-resolution that is over 10-fold beyond the intrinsic resolution of the system that one of ordinary skill would predict as being imposed by the physical constraints of the materials utilized in the manufacture of components in the system. The circuit of the invention may be embodied in a high-resolution constant-current stimulator neuroprosthesis neurostimulator chip. The circuit of the invention may be embodied in an electrical neuromodulation neuro stimulator chip for the generation of neurostimulation signals in a neuroprosthesis. The invention additionally comprises a method of rehabilitating an amputee by fitting the amputee with a tactile-sensitive neuroprosthesis comprising the neurostimulator chip of the present invention. A preferred neuroprosthesis is a prosthetic forearm and hand while an alternative embodiment preferred neuroprosthesis is a prosthetic hand.

### BRIEF DESCRIPTION OF THE DRAWINGS

FIGS. 1A and 1B are circuit diagrams that provide circuit schematics that show the basic reverse crossfire, or RXR, circuit components of the invention.

FIG. 2 is a four-part circuit diagram that provides circuit schematics of four alternative embodiments that achieve an identical neural signal output.

FIG. 3 is as distribution chart displaying the number of redundant configurations corresponding to individual output codes of various RXF structures.

FIGS. 4A, 4B, and 4C are graphical representations of varying degrees of configuration code diffusion.

FIGS. 5A and 5B are the graphical results of a Monte Carlo simulation of super-resolution created by the invention.

FIG. 6 is the graphical result of a Monte Carlo simulation of random mismatch ratio to achieve super-resolution.

FIG. 7 is a circuit diagram that provides a circuit schematic of a fully integrated neurostimulator design.

FIG. 8 is a tabular chart of typical physical measurements of transistors used in the RXF circuit.

FIG. 9 is a schematic flow-chart of the process of tuning transistor mismatch.

FIG. 10 is the graphical results of a Monte Carlo simulation of the effect of tuning on effective mismatch.

FIG. 11 is a flow chart and schematic illustration of a stimulator chip control strategy.

FIG. 12 is a micrograph of a neuromodulator chip of the invention.

FIGS. 13A and 13B are graphical representations of chip performance under varying mismatch management strategies.

FIG. 14 is a graphical representation of actual computed mismatch error under varying mismatch management strategies.

FIGS. 15A and 15B are graphical representations of a stimulator channel integral nonlinearity and of differential nonlinearity.

FIGS. 16A and 16B are graphical representations of the results of output measurements that demonstrate that decreasing current levels yield lowered distortion levels.

FIG. 17 is a graphical representation of charge mismatch as a function of charge-balancing capacity of a stimulator channel.

FIG. 18 is a graphical representation of the results of a stimulator's charge-balancing characteristics measured in a buffered saline solution.

FIG. 19 is a set of photo images of a prosthesis incorporating a neurostimulation module as part of the prosthesis complete system.

FIG. 20 is a graphic representation readout of current amplitude pulses generated by prosthesis tactile contact with a solid surface.

FIGS. 21A and 21B are a pair of graphic representations of before-and-after nerve signal quality following stimulation artifact removal.

### DETAILED DESCRIPTION OF THE DRAWINGS AND INVENTION

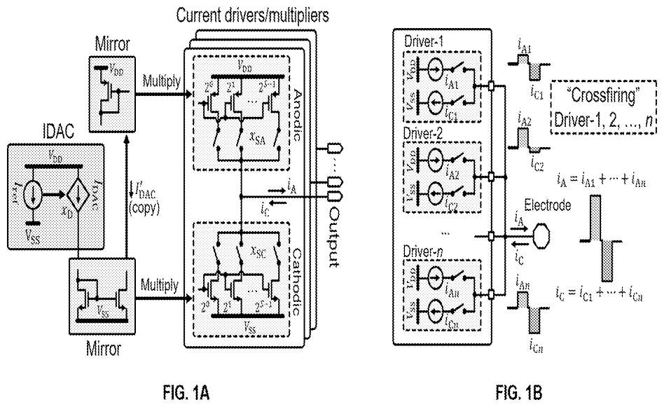

Turning first to FIGS. 1A and 1B, there is shown a preferred embodiment of the basic RXF circuitry components, so configured as to create a structure of information redundancy, it being understood herein that numerous alternative different internal configurations can produce the same type of desired output. An important consideration is that a circuit architecture as disclosed here will realize its desired output qualities with a significantly reduced requirement of physical components, as compared to the prior art.

FIG. 1A illustrates a preferred embodiment of a standard, biphasic, current-mode stimulator, which is comprised of a current digital-to-analog converter (IDAC) 101, current mirror circuits 102 and 103, and anodic (positive) 104, and cathodic (negative) 105 output current driver/multipliers. The drivers perform dual functions: firstly, the scaling of the IDAC current by a multiplying factor, and secondly, the driving of a constant current to an electrode. For each channel of a given embodiment, there is one IDAC that is shared among a plurality of discrete drivers. Subsequently, the anodic i_{A} and cathodic i_{C} output currents are expressed as follows:

i_{A}=I_{IDAC}·x_{SA}=I_{ref}·x_{D}·x_{SA}

i_{C}=I_{IDAC}·x_{SC}=I_{ref}·x_{D}·x_{SC}

Where I_{ref} is a fixed reference current, x_{SA} and x_{SC} are the multiplier codes, and x_{D} is the IDAC code.

FIG. 1B illustrates how an RXF structure is created by functionally connecting and combining, or as used in this disclosure, “cross firing” the output of two or more drivers 106, 107, and 108. Each driver contributes a small amount of current shown as pulse forms 109 for driver 1, 110 for driver 2, and 111 for driver n, which together constitute the input current i_{A} 112, which may be independently adjusted to generate a final desired stimulation pulse 113, and that constitutes the output current i_{C} 114. The timing of the pulses produced by different drivers must be synchronized, meaning that the anodic and cathodic currents are turned on and off at exact moments in time such that they will behave as a single stimulation channel. This synchronization can be easily achieved with an on-chip timing generator means, well known to those of ordinary skill in the art, that turns on, and turns off, the output from two or more drivers simultaneously at desired and calculated time internals so as to achieve synchronization of the outputs.

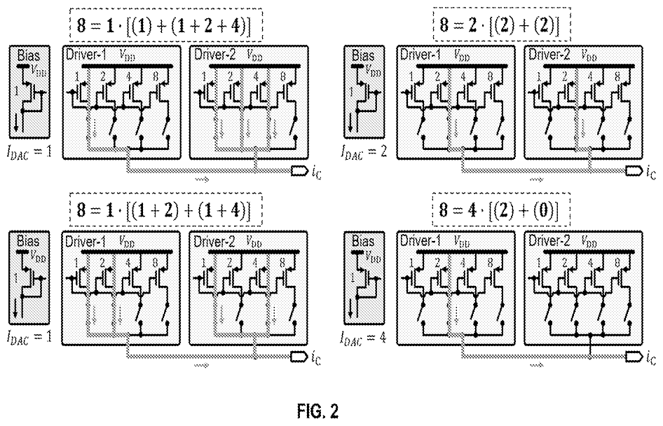

FIG. 2 illustrates examples of several alternative circuit configurations that produce the same theoretical output when there is no mismatch error. Here the IDAC's resolution is expressed as N_{D}=5 and the multiplier's resolution is expressed as N_{S}=4. These four illustrated configurations of different IDAC and multiplier values will generate the same output code i_{C}=8. The RXF structure is seen to be redundant because the same output code can be generated by numerous different configurations of the IDAC and multiplier. In practice, there may be up to tens to hundreds of distinct configurations associated with each output code. The varying configurations of the IDAC and multipliers exhibit complex mutual relationships that depend on the resolution capability of the IDAC, of the multipliers, and of the number of crossfire drivers.

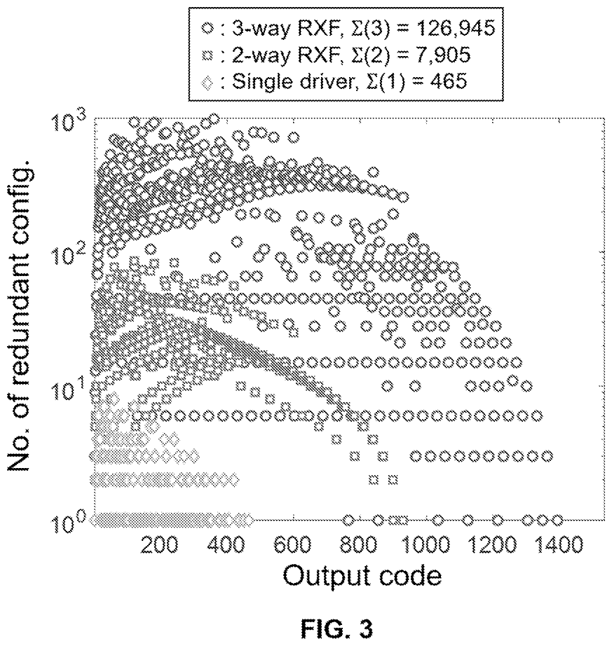

FIG. 3 illustrates a distribution of the number of redundant configurations 301 corresponding to each output code in a single driver, in this non-limiting example given as a 2-way RXF 302, and a 3-way RXF 303 structure across the sample space, compared to an apparatus having a single driver 304. Manufacturing additional crossfire drivers thus can be seen to extend the maximal output, and exponentially increase the level of redundancy, which is essential to achieve super-resolution. The total number of non-zero, distinct configurations in an n-way RXF structure with n crossfire drivers is expressed as follows:

Σ(n)=(2^{N}^{_{D}}−1)·[(2^{N}^{_{S}})ⁿ−1]

With each additionally added crossfire driver, the number of configurations (i.e., level of redundancy) grows exponentially, but the physical resources required, i.e., for example chip area, will only increase linearly. This is a significant improvement in improving signal output and quality without a parallel increase in apparatus resource, which would otherwise impose physical size constraints and energy consumption constraints on a neuromodulation device.

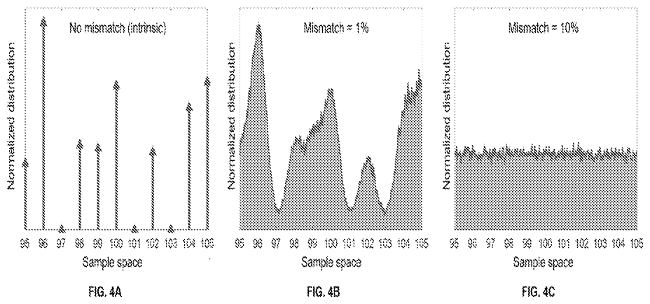

FIGS. 4A, 4B, 4C show a unique property of a redundant structure called “code diffusion” that enables super-resolution. The graphs show the distribution of the analog outputs produced by three different configurations of a 2-way RXF structure. The analog outputs are contained in a continuous sample space with values ranging from 0 to a maximum value of approximately 31·(15+15)=930 LSB where 1 LSB=I_{ref}. A least significant bit (LSB) is the bit position in a binary integer giving the units value, that is, determining whether the number is even or odd. The LSB is sometimes referred to as the low-order bit or right-most bit, due to the convention in positional notation of writing less significant digits further to the right. It is analogous to the least significant digit of a decimal integer, which is the digit in the ones (right-most) position. To model the random mismatch error, it is assumed that each unit-transistor of both the IDAC and the multipliers has a Gaussian distribution, and that the standard deviation is the mismatch ratio. When generating random samples, negative values are set to zero. FIG. 4A shows that, with no mismatch errors, redundant configurations generate the exact same analog outputs 401 that are centered at integral integer codes 402, e.g. [0, 1, 2, 3, . . . ]. Their distributions are represented by Dirac delta impulses with the weight equal to the number of configurations. FIG. 4B shows that, with small random mismatch errors, the actual values of different analog outputs begin to deviate from their original states and “diffuse” into the adjacent sample space 403. FIG. 4C shows that, with large mismatch errors, the actual values of different analog outputs have exponentially increased and completely diffused 404, and are now evenly distributed across the sample space.

Code diffusion allows for the generation of sub-integer codes, e.g. [0.1, 0.2, 0.3, . . . with a certain probability that is not normally possible. These sub-integer codes correspond to the sample space's finer partitions, so that there is thus an effective super-resolution that is beyond the baseline figure. For example, to achieve a (+1) super-resolution, redundant configurations that generate all the e.g. sub-integer codes of [0, 0.5, 1, 1.5, . . . ] must be found. To achieve (+2) super-resolution, the required sub-integer codes are [0, 0.25, 0.5, 0.75, 1, . . . ]. While identifying the correct configuration for every output code is an NP-hard optimization problem, it is only possible in an information redundant architecture such as RS. In computational complexity theory, a problem is NP-complete when: a brute-force search algorithm can solve it, and the correctness of each solution can be verified quickly, and the problem can be used to simulate any other problem with similar solvability; where RS is Reed-Solomon codes, which operate on a block of data treated as a set of finite field elements called symbols. RS codes are able to detect and correct multiple symbol errors. The probability of accomplishing this task is maximized when the codes distribute evenly across the sample space, as shown in FIG. 4C.

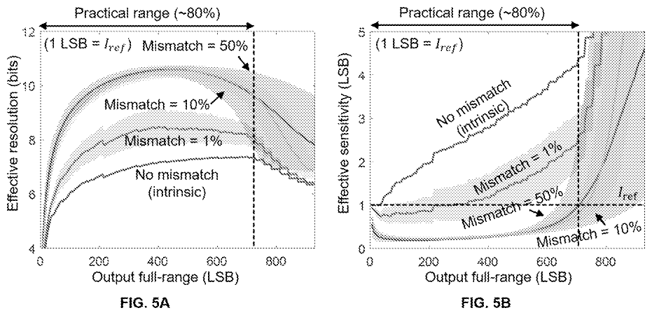

FIGS. 5A, and 5B illustrate Monte Carlo simulation results to evaluate the theoretical super-resolution that can be achieved with the present invention. Here, the mismatch error is applied to elements of both the IDAC and multiplier equally. In each simulation, the RXF structure is optimized by an automated brute-force approach, i.e., sorting through all the possible redundant configurations and finding the ones that generate the desired output with the least amount of error. We use the terms “effective resolution” and “effective sensitivity” to measure quantitative performance. The effective resolution is defined as the Shannon entropy, which is computed with respect to a targeted resolution (12 bits) is expressed as follows:

$${M}_{{N}_{x}} = {\sum }_{d = 0}^{{2}^{{N}_{x - 1}}} {\int }_{{θ}_{d}}^{{θ}_{d + 1}} {( {x}_{A} - \frac{d + 0.5}{{2}^{{N}_{x}}} )}^{2} {dx}_{A} and$$

$${H}_{{N}_{x}} = - {log}_{2} \sqrt{12 \cdot {M}_{{N}_{x}}}$$

where H_{N}_{ₓ} is the effective resolution (entropy) with respect to the targeted resolution Nₓ; M_{N}_{ₓ} is the normalized total mean-square-error integrated over each digital code d∈[0, 2^{N}^{ₓ}−1]; θ₀, θ₁, . . . are the corresponding analog outputs. The targeted resolution Nₓ is the reference upper-bound of the device's super-resolution, which is arbitrarily defined over the full-range. No matter how high the targeted resolution is defined, the effective resolution H_{N}_{ₓ} would converge to a maximum value. The effective sensitivity is defined as the smallest change in output current that could be accurately produced by the device. It is computed as follows:

$${S}_{{N}_{x}} = {I}_{ref} \cdot \frac{FR}{{2}^{{H}_{{N}_{x}}}}$$

where S_{N}_{ₓ} is the effective sensitivity; FR is the targeted output full-range. The metrics are evaluated for all values of the full-range from 0 to the maximum value of 930LSB.

FIG. 5A shows that RXF enables the achievement of an effective super-resolution that is practically always higher than the device's intrinsic resolution. The effective super resolution, at different values of output full-range, is highly correlated with the number of redundant configurations. The effective resolution in the first 0-50 LSB is low because there are not enough redundant configurations. The effective resolution reaches its highest point at approximately 50-80% (450-750 LSB) of the maximum full-range in which most of the redundant configurations are located. At the practical full-range of 80% (750LSB), the RXF structure achieves 2-3 bits super-resolution beyond the intrinsic baseline. The intrinsic baseline is the best resolution that can be attained with the prior art conventional structure and zero mismatches. Beyond this point, the resolution drops rapidly because the redundant configurations become sparsely distributed, thus there is insufficient redundancy. Moreover, the illustrated results clearly demonstrate a novel, unobvious, unique, and fundamental functional capability of the present invention, which is that mismatch error is utilized to actually enhance resolution. Unlike any previous design in the prior art, the proposed system becomes more accurate as and when the mismatch ratio increases from 0 to 10%. This is because a larger mismatch error leads to a more even distribution of redundant values across the sample space, which maximizes the probability of finding a configuration to generate the desired output. The effectiveness also becomes more consistent (i.e., smaller deviation) when the mismatch ratio increases from 0 to 10%. This shows that the super-resolution achieved by the present invention is high-yield, replicable, and does not rely on a specific random configuration.

FIG. 5B illustrates that similar functions of the invention are produced, in terms of the other parameter being used, namely effective sensitivity, which is proportional to the inverse of the effective resolution.

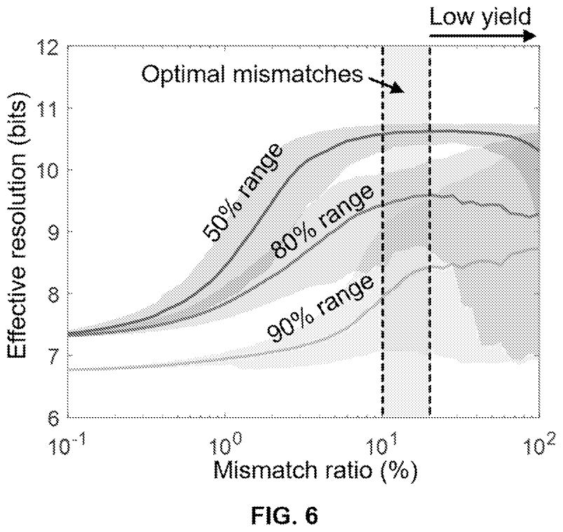

FIG. 6 illustrates Monte Carlo simulation results from evaluating the optimal range of random mismatch ratio to achieve super-resolution. The graph shows the effective resolution, computed at 50%, then 80%, then 90% ranges with different mismatch ratios of from 0.1-100%. The present invention demonstrates that in a novel, non-predicted, and surprising manner, contrary to the teachings of the prior art, a mismatch ratio within a range of about 10 to 20% yields the most preferred, optimal, super-resolution at most ranges. Above that range, not only is there is little significant additional boost to the effective resolution, but also, deviation starts to widen. This suggests a lower effective yield, since fewer samples are capable of achieving the desired super-resolution, and the structure may well become unreliable.

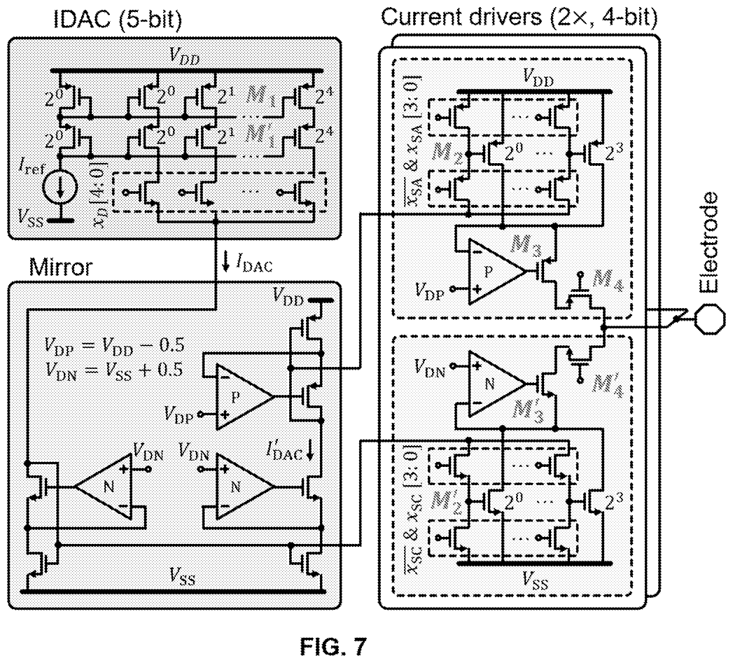

FIG. 7 illustrates the schematic of a fully-integrated neurostimulator design with 2-way RXF architecture n=2, N_{D}=5, N_{S}=4. The IDAC is shown at 701, the current mirror is at 702, and current drivers are at 703 and 704. The current mirrors and drivers are based on an op-amp assisted boosted-cascode current driver and current mirror. Here the design trades away or trades off device area in order to to achieve ultra-high-output impedance. The estimated output impedances are >1 GΩ at 1 mA (source) and >50 GΩ at −1 mA (sink). The output voltage range is set by V_{DN}=V_{SS}+0.5 (V) and V_{DP}=V_{DD}−0.5 (V), which results in compliance of ±4.5V. The reference current is generated by a voltage-to-current converter circuit, and the value is set to. I_{ref}=1.5 μA by V_{DD} and by an external resistor.

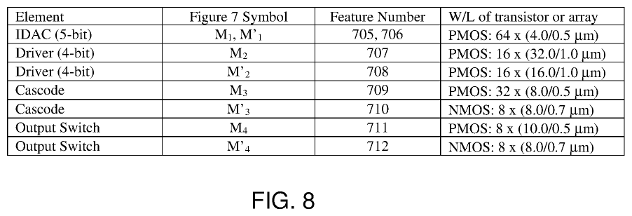

FIG. 8 shows, with respect to the circuit in FIG. 7, the width to length dimensions, in μm, of key transistors and transistor arrays illustrated in FIG. 7. Minimum feature-size transistors are used whenever possible (e.g., IDAC) to maximize the available mismatch error. Nevertheless, larger transistors are needed for the driver, the cascode, and the output switches, in order to meet the voltage-drop requirement for maintaining output compliance. The anodic circuits generally use larger transistors than their cathodic counterparts because the current drivability of P-channel metal-oxide-semiconductor (PMOS) is about 50% less than that of N-channel metal-oxide semiconductor (NMOS).

Proceeding contrary to conventional wisdom, the present invention utilizes a large level of transistor mismatch of approximately 10-20% as a desirable element of a most preferred design embodiment. The present invention's key advantage is its ability to convert mismatch error from a concerning and vexing problem into a desirable and useful means for achieving previously unachievable signal resolution and sensitivity, allowing designers of ordinary skill in the art to utilize smaller-sized components, and to relax physical layout constraints, which were engineering compromises that were required to be made in order to suppress mismatches in past design approaches. While the implication is that the RXF technique would work better in a deep submicron complementary metal-oxide semiconductor (CMOS) process with a large amount of mismatch, the random mismatch existing in the standard CMOS process may not be adequate. In situations where a “naturally” occurring mismatch is thought to be insufficient, it is now possible to increase the amount of error on purpose.

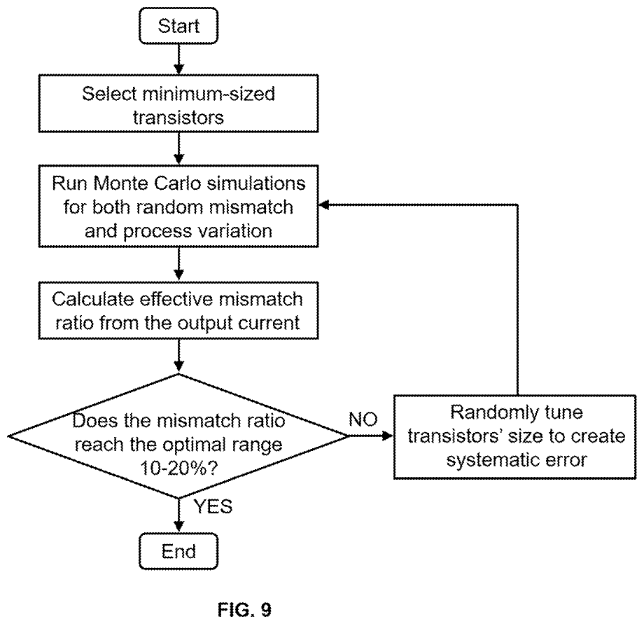

FIG. 9 illustrates a flow chart of a preferred procedure to tune a transistor mismatch ratio to achieve optimal super-resolution. Monte Carlo simulations can be used during the design process to evaluate the effective mismatch ratio. Random mismatch error (spread) is caused by variations and mismatches, while process (systematic) error (offset) is caused by non-ideal schematic and parasitic elements. If the random error in a design does not reach the most preferable level (i.e, 10-20%), then “artificial” mismatches may be created by arbitrarily tuning the individual unit transistors' size (W/L) by a range of from a few tens to a few hundred nanometers from the nominal value. The tuning values are randomly generated in a computer and manually added to each unit-transistor, (though this may influence the systematic error). The procedure is repeated with each bit of the IDAC and multiplier to the extent necessary. The procedure is ideally carried out with post-layout simulations, because the parasitic elements could result in creating additional systematic errors.

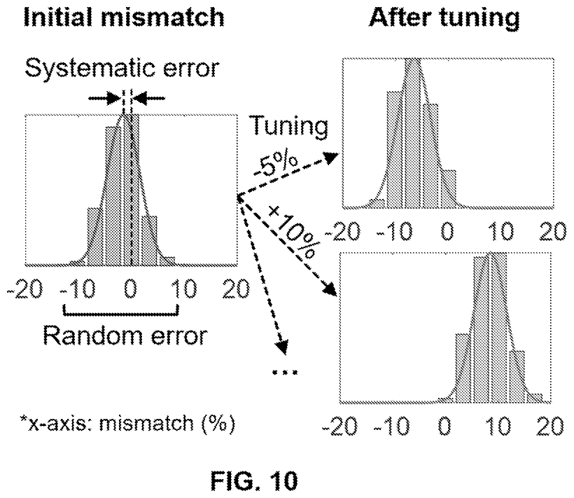

FIG. 10 illustrates the result of a Monte Carlo simulation, where the effective mismatch includes both the systematic error 1001 and random error 1002 components. The tuning process described above may introduce sought-after additional systematic errors, while not affecting the random error component.

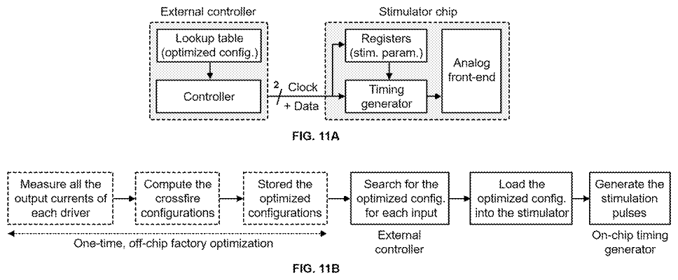

FIGS. 11A, and 11B show the present invention's stimulator control strategy, which consists of both an on-chip logic/memory process, and an off-chip logic/memory process. To achieve super-resolution, an RXF structure must be optimized. This is done with a one-time chip calibration procedure at the chip factory where all of the driver's output currents are measured. There are 2 (2⁵−1)·(2⁴−1)=930 non-zero values per driver to be measured, both cathodic and anodic. All the crossfire configurations are then computed. There are 2 (2⁵−1)·(2⁴·2⁴−1)=15,810 non-zero configurations per channel in a 2-way RXF structure. The optimized configuration associated with each desirable output current can be easily found by sorting through all the available values in a brute-force manner. While it is not an elegant solution, the optimization procedure only needs to be done once with the off-chip computation. The optimized configurations are then stored in an external lookup table. With a 10-bit effective resolution, the table size would be calculated to be 2 2¹⁰ (5 +4 +4)=26,624 bits (3.3 kB) per channel. An external controller maps each desirable output with the lookup table's optimized configuration during normal operation. The configuration is loaded into the stimulator chip via a transmission protocol consisting of a 10 MHz clock line and a data line.

The on-chip timing generator circuits produce stimulation pulses. This is essential to achieve a near-perfect synchronization of multiple drivers in a crossfire configuration. All of the stimulation parameters, such as pulse-width, IDAC, multiplier, polarity, and the like, are stored in integrated registers. The anodic and cathodic phases of a biphasic pulse can be independently configured to produce both symmetrical and asymmetrical stimulation with any ratio setting. New IDAC and multiplier configurations are loaded during the interphase delay. In a most preferred embodiment of the invention, a 16-bit register at a base clock of 10 MHz to control the pulse-width is used. This allows generating any timing from 0.01 msec to 6.56 msec with 0.1 μsec adjustment step. The adjustment step is also used to digitally compensate for the residual mismatch between the anodic and cathodic currents to ensure charge-balancing. This is achieved by tuning the anodic and cathodic pulse-width such that:

minimize|i_{A}·(t_{A}+Δt_{A})−i_{C}·(t_{C}+Δt_{C})|

The adjustment timings (Δt_{A}, Δt_{C}) are computed by an external controller based on the measured currents i_{A}, i_{C} and required pulse-width (t_{A}, t_{C}).

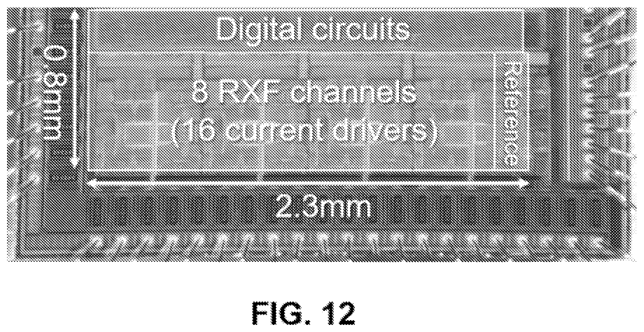

FIG. 12 shows a photomicrograph of a prototype chip of the most preferred embodiment of the invention, which was fabricated using the GlobalFoundries 0.18 μmBCDLite process. This embodiment utilizes isolated high-voltage LDMOS transistors, which are capable of supporting up to 30V. The disclosed chip contains 8 RXF channels (utilizing 16 current drivers) and occupies a core area of approximately 0.8 mm×2.3 mm. The chip's overall static power consumption is approximately 2.4 mW for the analog circuits at a 10V (±5V) supply and 1.6 mW for the digital circuits at a 1.8V supply. Most of the static power is generated from the high-voltage op-amps' bias current, which can be shut down during the interval that a channel is not in use.

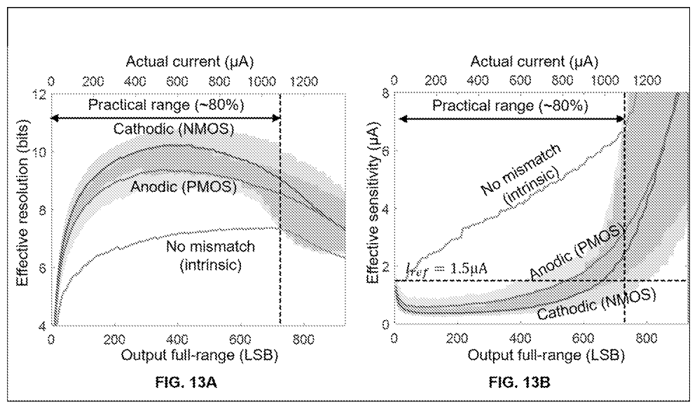

Chip measurement results. FIGS. 13A and 13B illustrate the measured results of the effective resolution and effective sensitivity for the above-described chip. The data were acquired from different channels and chips N_{Ch}=8, N_{IC}=10. The reference current was set to I_{ref}=1.5 μA, which translates to a practical output full-range of approximately 1.1 mA. The RXF technique of the present invention results in an effective super-resolution of 2-3 bits beyond the intrinsic baseline, conforming with the theoretical analysis. The effective sensitivity is well below the reference current across most of the output full-range, which was not possible to achieve in any prior art approach.

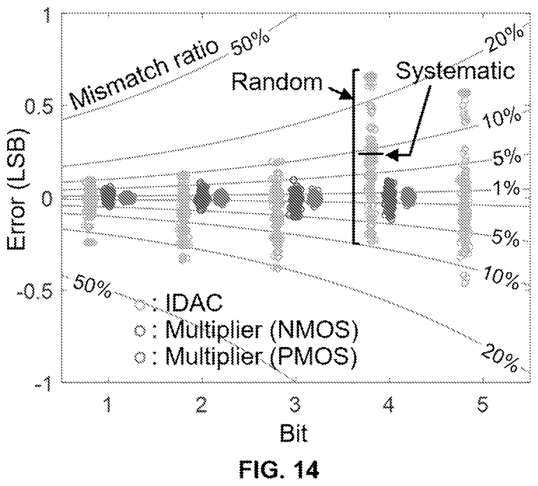

FIG. 14 illustrates the actual mismatch error computed from the measured current outputs for each bit of the IDAC and multiplier. The IDAC exhibits desirable random and systematic error, falling within the optimal 10-20% range.

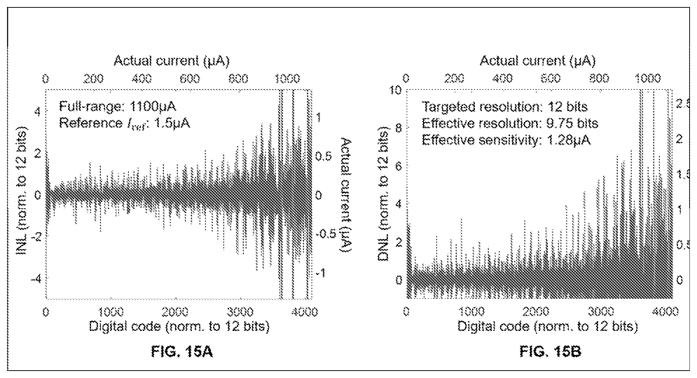

FIGS. 15A and 15B illustrate the measured integral nonlinearity (INL) and differential nonlinearity (DNL) of a stimulator channel. Here both the x-axis and y-axis are normalized to the targeted resolution of 12 bits over the 1.1 mA full range. The codes were optimized so that the outputs are always monotonic. The measured channel achieves an effective super-resolution of 9.75 bits and effective sensitivity of 1.28 μA. Unlike a conventional ADC/DAC, the INL and DNL of an RXF device are not symmetrical. Lower digital codes are more accurate because they contain more redundant configurations. The large spikes of INL/DNL data in higher digital codes (3500-4000 LSB) and the brief peak in the area of 0-50 LSB are associated with regions where there are not enough redundant configurations and their distribution is accordingly sparse.

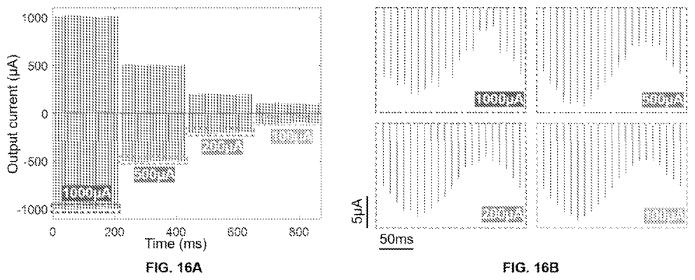

FIG. 16A illustrates examples of measured output current using, for example, a 1 kΩ resistive load. In this test, the chip generated a train of biphasic stimulation pulses at various output current levels of 100, 200, 500, and 1000 μA. Each pulse was accurately modulated to produce a sinusoidal waveform with a 5 μA ac amplitude. FIG. 16B zooms in to show greater detail of the pulse trains. The results indicate that the output at lower current levels is more accurate than the output at higher current levels, i.e., the sinusoidal waveforms of 100 or 200 μA are visibly less distorted than the sinusoidal wave forms of 500 or 1000 μA. Nevertheless, the waveform deviation at 1000 μA level is still within an accuracy of ±1 μA.

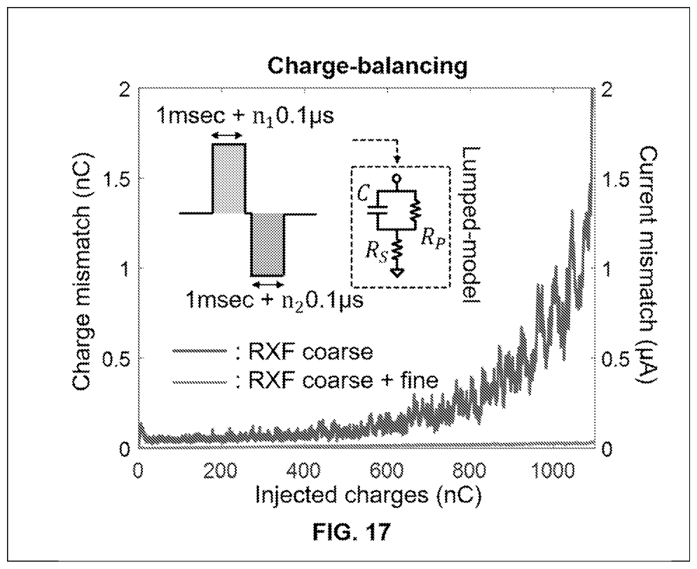

FIG. 17 illustrates a detail of the measured charge-balancing characteristics of a stimulator channel, previously alluded to in FIG. 1B. In this embodiment there were used a 1 msec pulse-width, anodic (positive) leading pulses, cathodic (negative) trailing pulses, and a lumped electrode model expressed as as C=0.5 μF, R_{S}=1 kΩ, R_{P}=10 MΩ. There are two levels of charge-balancing, coarse and fine, both of which are digitally calculated by an external controller based on the measured currents and optimized configurations. The coarse calibration involves selecting the optimal IDAC and multiplier configuration of the second phase with a current amplitude matching the first phase. This process is done during the one-time factory optimization based on the measured current of every RXF configuration. Both the IDAC and multiplier values are adjusted during the inter-phase delay and require less than 10 μsec settling time. The fine calibration involves digitally tuning the second phase's pulse-width with 0.1 μsec steps to further compensate for any residual mismatch between the absolute amplitudes of the anodic and cathodic currents. With coarse calibration alone, the current mismatch is less than 0.2% across the entire output full-range. Again, lower current levels have more accurate matching. When combined with fine calibration, the overall charge mismatch is reduced to an insignificant degree ($<0.005%$).

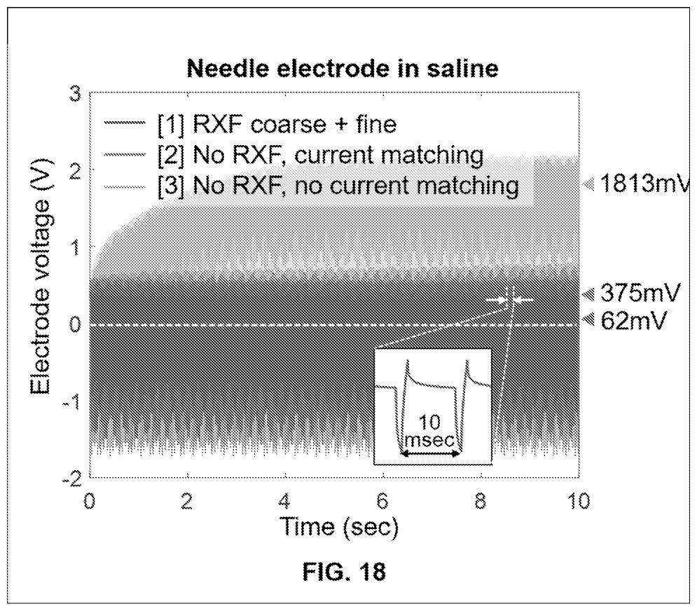

FIG. 18 illustrates the results of an aqueous environment experiment to verify the stimulator's charge-balancing characteristics in an aqueous saline solution. A pair of stainless-steel needle electrodes were submerged in a phosphate-buffered saline (PBS) preparation. The electrode's impedance was measured at 7.4 kΩ at 1 kHz. The stimulus is a train of cathodic leading, biphasic, symmetric pulses, with 1 msec pulse-width, and a targeted 200 μA current amplitude. The stimulation rate was 100 Hz, which approaches the upper bound of a most preferred embodiment application for the device. The 10 msec pulse-to-pulse spacing was selected since that rate does not allow sufficient time for the electrode to naturally discharge. In the first measurement, RXF with both coarse and fine calibration was used, and after 1000 pulses, the electrode's residual voltage stabilized at about 62 mV. The estimated current amplitudes were estimated and set at [−200.1, 200.0]μA, and 0.5 μsec was added to the second phase. In the second measurement, only one multiplier (but with no RXF) was used, to match the anodic and cathodic current with available configurations, and the residual voltage reached 375 mV and continued to increase. The estimated current amplitudes are [−201.2, 201.6]μA. In the third measurement, neither RXF nor current matching was used, and the residual voltage quickly reached 1813 mV, saturating the electrode interface as water began to undergo electrolysis. Current amplitudes were [−223.2, 230.7]μA.

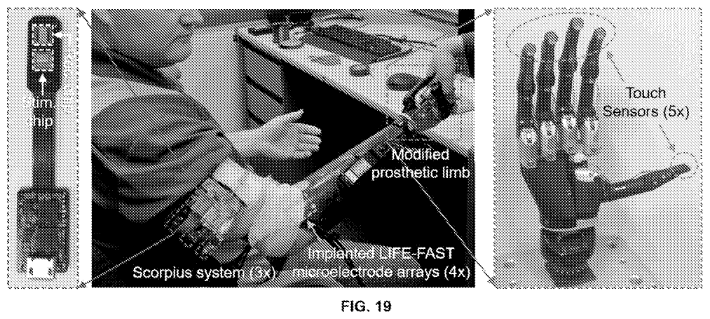

FIG. 19 photographically illustrates a neuroprosthesis experimental setup, which demonstrates the need for a high-resolution, fully-integrated neurostimulator, and this need is met by the chip architecture utilized in the present invention. The experiment is designed to restore somatosensation in a transradial amputee using electrical microstimulation while simultaneously acquiring nerve signals and generating new microstimulations that are re-calibrated in response to the processing of acquired nerve signals, in order to control a prosthetic hand's movements. A most preferred embodiment of a neurostimulator chip component is the essential part of the Scorpius neuromodulation system, a proprietary system known to those of ordinary skill in the art, that has both recording, and stimulation functions. The design and specifications of the Scorpius system are reported in Nguyen & Xu et al. A Bioelectric Neural Interface Towards Intuitive Prosthetic Control For Amputees. Journal of Neural Engineering, 17(6), 066001. (2020), the entire disclosure of which is incorporated herein by reference. Three Scorpius devices were used in the setup shown in FIG. 19, which can simultaneously address 24 independent stimulation channels. Four longitudinal intrafascicular electrode (LIFE) arrays were implanted into the patient using the microsurgical fascicular targeting (FAST) technique. The FAST-LIFE microelectrodes target discrete fascicles in the median and ulnar nerves. The design and characteristics of the electrodes are reported by Cynthia K. Overstreet et al Fascicle specific targeting for selective peripheral nerve stimulation. J. Neural Eng. 16 066040 (2019), the entire disclosure of which is incorporated herein by reference. The electrode's wires penetrated through the patients' skin and were connected to the Scorpius devices via standard Omnetics nano-connectors. The prosthesis was a heavily modified i-Limb Access hand (Touch Bionics, Livingston, UK). The prosthetic hand was equipped with touch sensors (Interlink Electronics, CA, USA) at the fingertips. In this most preferred embodiment of the present inventions, the hand's driver was replaced with a customized controller using the ESP32-WROOM-32 module (Espressif Systems, Shanghai, China). The ESP32 sampled force readouts from the sensors at 50 Hz and relayed data to the host server via Bluetooth. The host server used the sensor readouts to modulate the stimulation pulses' current amplitude to create various levels of touch sensation in the patient.

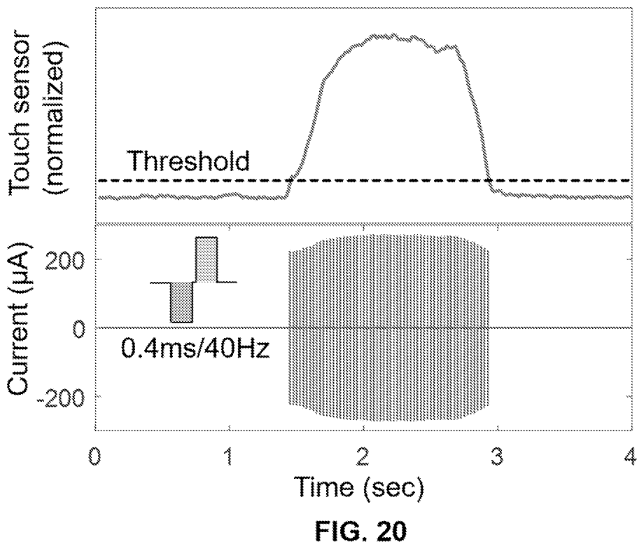

Turning now to FIG. 20, there is shown the resultant stimulation pattern as a function of elapsed time, as the prosthesis touched and released an object, which stimulation signal was applied to an electrode with clear sensory precepts acquired from previous mapping experiments. The stimulation pattern was a train of cathodic-leading, biphasic, symmetric pulses with a 0.4 msec pulse-width, at a 40 Hz rate. The current amplitude was modulated to be proportional to the applied force. The pulses were only generated when the sensor readout was above a certain threshold. The neurostimulator's super-resolution was essential for modulating the current amplitude with 1 μA accuracy at any given threshold within a 10-1100 μA range. In one particular electrode embodiment shown in FIG. 20, the current amplitude ranged from 220 μA to 280 μA. The lower threshold 220 μA is the smallest current amplitude at which the patient can just barely begin to perceive sensation, while the higher threshold 280 μA is the highest current amplitude at which the patient can comfortably receive and tolerate signals without experiencing pain. Thus, it is essential to produce an accurate current amplitude within this range in order to deliver continuous and desirable sensory feedback from light to strong touch. It also noteworthy that the current thresholds vary widely across electrodes, even for those within the same microelectrode array. The thresholds may be as low as 15 μA and as high as 1000 μA. However, for any specific electrode, the working range (i.e., lower to higher threshold) is most preferably and 50-100 μA.

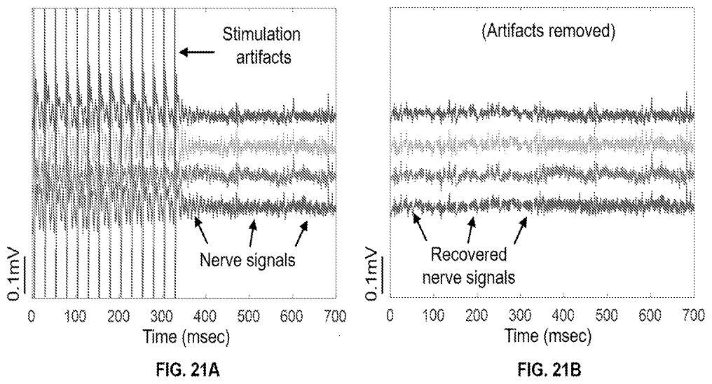

FIG. 21A and 21B illustrates neural recordings acquired by the above described Scorpius system while the amputee patient flexed one of the prosthetic “phantom” fingers, and the resultant stimulation signal from the prosthetic finger's tip touchpad was delivered to an adjacent electrode. A charge-balanced neurostimulator also played an important role in motor decoding experiments with simultaneous somatosensory feedback. The data show that the stimulation artifacts overlapped with the nerve signals. Charge-balancing helps reduce the impact of artifacts and prevents long-term charge accumulation, which could hinder the recorder's operation. The results show that the artifacts may be removed to recover most of the nerve data for decoding the amputee's motor intentions. The artifacts are removed offline using the template matching method for demonstration purposes. A brief duration of 2-3 msec at the onset of each stimulation pulse is removed and replaced with a straight line because the recorder's input is fully saturated.

While the above description contains much specificity, these should not be construed as limitations on the scope of any embodiment, but as exemplifications of the presented embodiments thereof. Many other alternative embodiments and variations are possible within the teachings of the various embodiments. While the invention has been described with reference to exemplary embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention will not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are, unless otherwise stated, used in a generic and descriptive sense only and not for purposes of limitation, the scope of the invention therefore not being so limited. Moreover, the use of the terms first, second, etc. do not denote any order or hierarchy of importance, but rather the terms first, second, etc. are used to distinguish one element from another. Furthermore, the use of the terms a, an, etc. do not denote a limitation of quantity, but rather denote the presence of at least one of the referenced items.

While the invention has been described, exemplified, and illustrated in reference to certain preferred embodiments thereof, those skilled in the art will appreciate that various changes, modifications, and substitutions can be made therein without departing from the spirit and scope of the invention. It is intended, therefore that the invention be limited only by the scope of the claims which follow, and that such claims be interpreted as broadly as is reasonable.

What is claimed is:

1. A neurostimulator system, comprising:
at least one digital-to-analog converter configured to receive an analog peripheral nervous system electrical signal from a patient and to convert said analog signal into a corresponding digital signal;
at least two current mirror circuits configured to receive digital electrical signals from said digital-to-analog converter and to provide mirrored current to at least two additional circuit components;
at least two or more current drivers, at least one being an anodic output current driver, and at least one being a cathodic output current driver, said drivers being configured to scale the current signals received from said mirror circuits by a multiplying factor, and further configured to driving the constant current to at least one output electrode;
wherein said outputs of said two or more current drivers are configured so as to create a combined, crossfiring, output of said current drivers that produces a redundant sensing structure that produces accurate current pulses with an effective super-resolution accuracy beyond ordinary limitations imposed by physical constraints of materials in said system.

2. The system as claimed in claim 1, wherein said redundant structure is configured so as to achieve a super-resolution signal accuracy outcome by applying the effects of random mismatch error function to said system, with the proviso that mismatch avoidance and mismatch compensation functions are not applied in achieving said super-resolution signal accuracy outcome.

3. The system as claimed in claim 2, wherein said random mismatch error function is configured so as to select and tune transistor size to achieve a desired mismatch ratio of 10% to 20%.

4. The system as claimed in claim 3, additionally comprising an on-chip timing generator.

5. The system as claimed in claim 4, additionally comprising both on-chip and off-chip components configured so as to retrievably store calculated optimal transistor configurations obtained through foreground calibration, and which configurations can be retrieved and read by said on-chip timing generator so as to produce signal output with super resolution accuracy.

6. The system as claimed in claim 5, wherein said on-chip component is a memory chip component of said system.

7. The system as claimed in claim 5, where said off-chip component is a look-up table component of said system.

8. The system as claimed in claim 1, additionally comprising an external controller configured so as to ensure charge-balancing that is achieved by digital compensation for residual mismatch between said anodic and cathodic currents.

9. The system as claimed in claim 8, wherein said charge balancing is further characterized as being coarse level charge balancing.

10. The system as claimed in claim 8, where said charge balancing is further characterized as being fine level charge balancing.

11. The system as claimed in claim 1, configured so as to modulate the neurostimulation intensity of said crossfiring redundant signal output to create various levels of somatosensorial signal outputs of from light to strong touch in real time in a neuroprosthesis device.

12. The system as claimed in claim 2, wherein said random mismatch error function is configured so as to select and tune diode size to achieve a desired mismatch ratio.

13. The system as claimed in claim 2, wherein said random mismatch error function is configured so as to select and tune resistor size to achieve a desired mismatch ratio.

14. The system as claimed in claim 2, wherein said random mismatch error function is configured so as to select and tune capacitor size to achieve a desired mismatch ratio.

15. The system as claimed in claim 2, whereby application of the effects of random mismatch error function in said system is configured so as to be applied to extremely large mismatches to achieve super-resolution over 10-fold beyond intrinsic resolution of said design imposed by physical constraints of materials in said system.

16. A high-resolution constant-current stimulator neuroprosthesis neurostimulator chip, comprising:
at least one digital-to-analog converter configured to receive an analog peripheral nervous system electrical signal from a patient and to convert said analog signal into a corresponding digital signal;
at least two current mirror circuits configured to receive digital electrical signals from said digital-to-analog converter and to provide mirrored current to at least two additional circuit components;
at least two or more current drivers, at least one being an anodic output current driver, and at least one being a cathodic output current driver, said drivers being configured to scale the current signals received from said mirror circuits by a multiplying factor, and further configured to driving the constant current to at least one output electrode;
wherein said outputs of said two or more current drivers are configured so as to create a crossfiring, combined output of said current drivers that produces a redundant structure to produce accurate current pulses with an effective super-resolution beyond limitations of physical constraints of mismatch error in materials in said system.

17. An electrical neuromodulation neurostimulator chip for the generation of neurostimulation signals in a neuroprosthesis, wherein said chip comprises:
at least one digital-to-analog converter configured to receive an analog peripheral nervous system electrical signal from a patient and to convert said analog signal into a corresponding digital signal;
at least two current mirror circuits configured to receive digital electrical signals from said digital-to-analog converter and to provide mirrored current to at least two additional circuit components;
at least two or more current drivers, at least one being an anodic output current driver, and at least one being a cathodic output current driver, said drivers being configured to scale the current signals received from said mirror circuits by a multiplying factor, and further configured to driving the constant current to at least one output electrode;
wherein said outputs of said two or more current drivers are configured so as to create a crossfiring, combined output of said current drivers that produces a redundant structure to produce accurate current pulses with an effective super-resolution beyond limitations of physical constraints of materials in said system.

18. A method of rehabilitating an amputee by fitting said amputee with a tactile-sensitive neuroprosthesis comprising the neurostimulator chip of claim 17.

19. The method of claim 18, wherein said neuroprosthesis is a prosthetic forearm and hand.

20. The method of claim 18, wherein said neuroprosthesis is a prosthetic hand.
